(19) United States
(12) Patent Application Publication
(10) Pub. No.: US 2025/0237635 A1
(43) Pub. Date: Jul. 24, 2025

(54) METHOD FOR EVALUATING CATALYST LAYER FORMATION TRANSFER FILM

(71) Applicant: HONDA MOTOR CO., LTD., Tokyo (JP)

(72) Inventor: Fumiyoshi KUROSU, Saitama (JP)

(21) Appl. No.: 19/002,926

(22) Filed: Dec. 27, 2024

(30) Foreign Application Priority Data
Jan. 24, 2024 (JP) 2024-008891

(51) Int. Cl. G01N 33/00; G01N 3/58; H01M 4/88

(52) U.S. Cl. CPC G01N 33/0096; G01N 3/58; H01M 4/881

(57) ABSTRACT

A method for evaluating a catalyst layer formation transfer film is a method for evaluating a catalyst layer formation transfer film stacked on a base and transferred to form a catalyst layer of a fuel cell, which includes measuring a horizontal force by a SAICAS method while moving a cutting blade, and obtaining the maximum value of the horizontal force until the catalyst layer formation transfer film is broken after the cutting blade has been inserted into the catalyst layer formation transfer film and evaluating handleability upon transfer of the catalyst layer formation transfer film based on the maximum value of the horizontal force.

This application is based on and claims the benefit of priority from Japanese Patent Application No. 2024-008891, filed on 24 Jan. 2024, the content of which is incorporated herein by reference.

### BACKGROUND OF THE INVENTION

### Field of the Invention

The present invention relates to a method for evaluating a catalyst layer formation transfer film.

### Related Art

In recent years, in order to ensure that more people have access to reasonable, reliable, sustainable, and advanced energy, research and development has been conducted on a fuel cell contributing to enhancement of energy efficiency. Generally, the fuel cell has a membrane-electrode assembly (MEA) including an anode-side catalyst layer and a cathode-side catalyst layer facing each other through an electrolyte film. As a method for manufacturing the membrane-electrode assembly, a catalyst layer formation transfer film is transferred onto the electrolyte film. In order to avoid a failure in transfer of the catalyst layer, studies have been conducted on an evaluation method capable of evaluating occurrence of the failure from the state of the catalyst layer formation transfer film. For example, studies have been conducted on an evaluation method in which, when a cutting blade reaches an interface between a transfer film and a base on which the transfer film is provided, a vertical force and a horizontal force are measured by a SAICAS method and occurrence of a failure in transfer is predicted from a result obtained by dividing the vertical force value by the horizontal force value (see, for example, Japanese Unexamined Patent Application, Patent Document 1).
Patent Document 1: Japanese Unexamined Patent Application, Publication No. 2020-173905

### SUMMARY OF THE INVENTION

One object of the technique related to the fuel cell is improvement in productivity. Thus, an evaluation method has been demanded, which is capable of evaluating occurrence of a failure in transfer of a catalyst layer with favorable accuracy even in a case where various catalyst layer formation transfer films are used. However, in the conventional evaluation method using the SAICAS method, when the cutting blade reaches the interface between the catalyst layer formation transfer film and the base on which the catalyst layer formation transfer film is provided, the vertical force and the horizontal force are measured, and for this reason, such a method cannot be applied to a case where the catalyst layer formation transfer film is broken before the cutting blade reaches the interface.

The present invention has been made in view of the above-described situation, and an object thereof is to provide a method for evaluating a catalyst layer formation transfer film, which is capable of evaluating handleability upon transfer, such as occurrence of a failure in transfer of a catalyst layer, with favorable accuracy. Thus, the present invention contributes to an improvement in energy efficiency.

The present inventor(s) has found that the above-described problem can be solved by evaluating the handleability upon transfer using the maximum value of the horizontal force until the catalyst layer formation transfer film is broken after the measurement cutting blade has been inserted into the catalyst layer formation transfer film from the data on the horizontal force measured by the SAICAS method while the cutting blade is moving, and has arrived at the present invention.

(1) A method for evaluating a catalyst layer formation transfer film stacked on a base and transferred to form a catalyst layer of a fuel cell includes measuring a horizontal force by a SAICAS method while moving a cutting blade, and obtaining the maximum value of the horizontal force until the catalyst layer formation transfer film is broken after the cutting blade has been inserted into the catalyst layer formation transfer film and evaluating handleability of the catalyst layer formation transfer film upon transfer of the catalyst layer formation transfer film based on the maximum value of the horizontal force.

According to the method for evaluating the catalyst layer formation transfer film in (1), the maximum value of the horizontal force until the catalyst layer formation transfer film is broken after the cutting blade has been inserted into the catalyst layer formation transfer film is used from the data on the horizontal force measured while the cutting blade is moving, and therefore, even if the catalyst layer formation transfer film is easily breakable, the handleability upon transfer can be predicted with favorable accuracy.

(2) The method for evaluating the catalyst layer formation transfer film according to (1), in which evaluating the handleability upon transfer includes an assessment of the presence or absence of a flake and a breakage of the catalyst layer formation transfer film immediately after the catalyst layer formation transfer film has been transferred onto an electrolyte film of the fuel cell.

According to the method for evaluating the catalyst layer formation transfer film in (2), from the above-described horizontal force, the presence or absence of the flake and the breakage of the catalyst layer formation transfer film immediately after the catalyst layer formation transfer film has been transferred onto the electrolyte film of the fuel cell can be predicted.

(3) The method for evaluating the catalyst layer formation transfer film according to (2), in which evaluating the handleability upon transfer further includes an assessment of the presence or absence of a flake and a breakage of the catalyst layer formation transfer film when an external torsional force is applied to the catalyst layer formation transfer film.

According to the method for evaluating the catalyst layer formation transfer film in (3), from the above-described horizontal force, the presence or absence of the flake and the breakage of the catalyst layer formation transfer film when the external torsional force is applied to the catalyst layer formation transfer film can be predicted.

According to the present invention, the method for evaluating the catalyst layer formation transfer film can be provided, which is capable of evaluating the handleability upon transfer, such as occurrence of the failure in transfer of the catalyst layer, with favorable accuracy.

### BRIEF DESCRIPTION OF THE DRAWINGS

FIG. 1 is a sectional view of one example of a transfer material having a catalyst layer formation transfer film to be evaluated in a method for evaluating the catalyst layer formation transfer film according to one embodiment of the present invention;

FIG. 2 is a sectional view showing a state in which the catalyst layer formation transfer film is broken in the method for evaluating the catalyst layer formation transfer film according to one embodiment of the present invention; and

FIG. 3 is a graph showing data on the horizontal force of the catalyst layer formation transfer film measured in Example 1.

### DETAILED DESCRIPTION OF THE INVENTION

Hereinafter, a method for evaluating a catalyst layer formation transfer film according to one embodiment of the present invention will be described with reference to the attached drawings. Note that the embodiment described below is an example of the present invention and the present invention is not limited to the embodiment below.

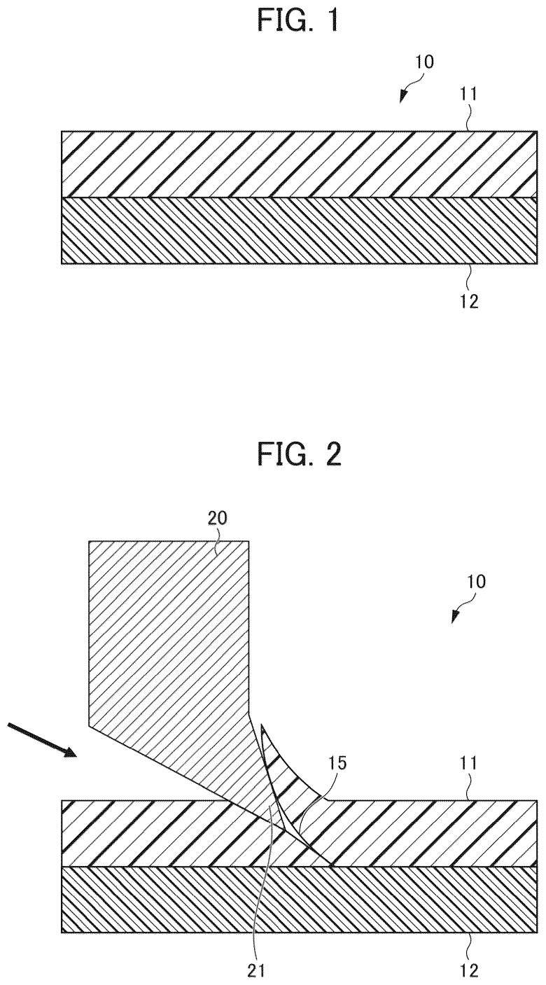

FIG. 1 is a sectional view of one example of a transfer material having a catalyst layer formation transfer film to be evaluated in the method for evaluating the catalyst layer formation transfer film according to one embodiment of the present invention.

As shown in FIG. 1, the transfer material 10 is a stack of the catalyst layer formation transfer film 11 and a base 12. The peel strength of the catalyst layer formation transfer film 11 may be within a range of 0.01 N/cm or more and 0.2 N/cm or less.

The base 12 is a member supporting the catalyst layer formation transfer film 11. The base 12 to be used may be, but is not limited to, a base used for a conventional transfer material. The catalyst layer formation transfer film 11 is transferred onto a surface of an electrolyte film of a fuel cell to form a catalyst layer of the fuel cell. The catalyst layer may be a cathode-side catalyst layer or an anode-side catalyst layer. The catalyst layer formation transfer film 11 is broken with a cutting blade used for a SAICAS method before the cutting blade reaches an interface between the base 12 and the catalyst layer formation transfer film 11. The catalyst layer formation transfer film 11 to be used may be, but is not limited to, one used for a catalyst layer of a conventional fuel cell.

In the method for evaluating the catalyst layer formation transfer film in the present embodiment, a horizontal force is measured by the SAICAS method while the cutting blade is moving. From data on the measured horizontal force, the maximum value of the horizontal force until the catalyst layer formation transfer film is broken after the measurement cutting blade has been inserted into the catalyst layer formation transfer film is obtained.

FIG. 2 is a sectional view showing a state in which the catalyst layer formation transfer film is broken. As shown in FIG. 2, in the surface and interfacial cutting analysis system (SAICAS) method, the cutting blade 20 with a pointed tip end 21 is inserted into a surface of the catalyst layer formation transfer film 11, and moves diagonally downward (direction indicated by an arrow in FIG. 2) at a certain speed. The horizontal force is a force provided from the catalyst layer formation transfer film 11 to the cutting blade 20 in the horizontal direction. In the present embodiment, before the cutting blade 20 reaches the interface between the catalyst layer formation transfer film 11 and the base 12, a breakage 15 is formed in the catalyst layer formation transfer film 11. The breakage 15 in the catalyst layer formation transfer film 11 can be checked by observation of the section of the catalyst layer formation transfer film 11 after measurement of the horizontal force, for example.

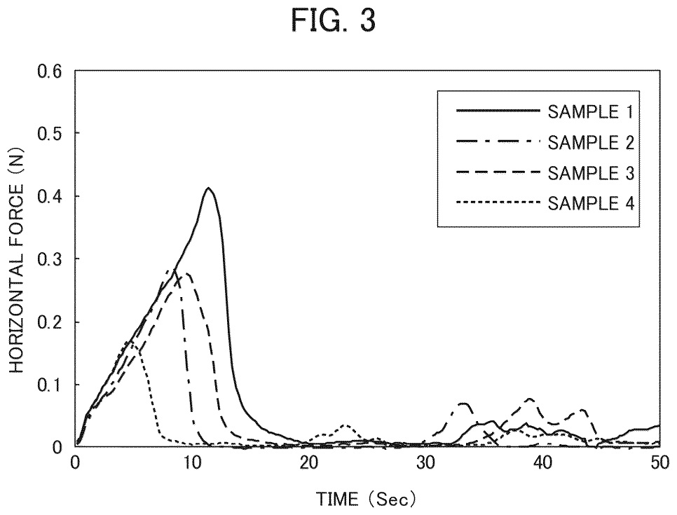

FIG. 3 is graph showing the data on the horizontal force of the catalyst layer formation transfer film measured in Example 1 described later. In the graph of FIG. 3, the horizontal axis represents time after insertion of the cutting blade 20, and the vertical axis represents the horizontal force. The graph of FIG. 3 shows that the horizontal force increases over time, i.e., as the insertion depth of the cutting blade 20 increases, and rapidly decreases thereafter. The point at which the horizontal force rapidly decreases is a point when the catalyst layer formation transfer film 11 is broken. The maximum value of the horizontal force is normally a horizontal force immediately before the catalyst layer formation transfer film 11 is broken.

In the method for evaluating the catalyst layer formation transfer film in the present embodiment, handleability upon transfer of the catalyst layer formation transfer film 11 is evaluated based on the maximum value of the horizontal force. An evaluation criterion changes due to, e.g., affinity between the catalyst layer formation transfer film 11 and the electrolyte film onto which the catalyst layer formation transfer film 11 is transferred, and for this reason, cannot be set uniformly. However, a case where the maximum value of the horizontal force is 0.2 N or more can be normally taken as acceptable.

According to the method for evaluating the catalyst layer formation transfer film in the present embodiment configured as described above, the maximum value of the horizontal force until the catalyst layer formation transfer film 11 is broken after the cutting blade 20 has been inserted into the catalyst layer formation transfer film 11 is used from the data on the horizontal force measured while the cutting blade 20 is moving, and therefore, even if the catalyst layer formation transfer film 11 is easily breakable, the handleability such as occurrence of a failure in transfer onto the electrolyte film can be predicted with favorable accuracy. For example, from the above-described horizontal force, the presence or absence of a flake and a breakage of the catalyst layer formation transfer film immediately after the catalyst layer formation transfer film has been transferred onto the electrolyte film of the fuel cell can be predicted. Further, from the above-described horizontal force, the presence or absence of a flake and a breakage of the catalyst layer formation transfer film when an external torsional force is applied to the catalyst layer formation transfer film can be predicted.

### EXAMPLES

### Example 1

Transfer materials of samples 1 to 4 were prepared. The transfer material is a stack of a catalyst layer formation transfer film and a base.

For the catalyst layer formation transfer film of each sample, a horizontal force was measured by the SAICAS method. Results are shown in FIG. 3, and the maximum horizontal force is shown in Table 1 below. The horizontal force was measured using a SAICAS (DN-GS type manufactured by DAIPLA WINTES CO., LTD.). As a cutting blade, a ceramic blade having a blade width of 1 mm, a rake angle of 20 degrees, and a clearance angle of 10 degrees and made of borazon was used. The following measurement conditions were employed: a measurement mode: a constant speed mode, a vertical speed: 0.5 μm/sec, and a horizontal speed: 5 μm/sec. Note that as a result of observation of the section of the catalyst layer formation transfer film with a scanning electron microscope (SEM) after measurement of the horizontal force, all the catalyst layer formation transfer films of the samples 1 to 4 were broken.

For the catalyst layer formation transfer film of each sample, a peel strength was measured. Results are shown in Table 1 below. The peel strength was measured by a 90-degree peel strength test. The transfer material was cut into a length of 100 mm and a width of 20 mm, and a peel strength measurement sample was produced. Using a double-sided tape (5000NS-25 manufactured by Nitto Denko Corporation), the peel strength measurement sample was bonded to a test device table with the catalyst layer formation transfer film on the lower side and the base on the upper side. The base of the peel strength measurement sample was peeled at a speed of 300 mm/min, and the 90-degree peel strength thereof was measured. As a result of observation of a surface of the peeled base, no catalyst layer formation transfer film remained on the base of any of the samples 1 to 4. Thus, it was confirmed that the peel mode of the catalyst layer formation transfer film by the 90-degree peel strength test is interfacial peeling.

Handleability upon transfer of each sample was evaluated by the following method. Results are shown in Table 1 below.

### (Evaluation of Handleability Upon Transfer)

A stack obtained in such a manner that the catalyst layer formation transfer film of the sample is stacked on a surface of an electrolyte film was disposed in a press through a cushion material, and was flattened by pressing under conditions where a temperature is 136° C., a pressure is 30 kgf/cm², and a pressing time is 5 sec. In this manner, the catalyst layer formation transfer film was pressure-bonded to the electrolyte film, and thereafter, the base of the sample was peeled off. Then, an electrolyte film-catalyst layer formation transfer film stack in which the catalyst layer formation transfer film is transferred onto the surface of the electrolyte film was obtained.

The surface of the catalyst layer formation transfer film immediately after transfer was visually observed, and the presence or absence of a flake and a breakage of 1 mm or more was checked. As a result, a case where the number of flakes and breakages per 100 cm² is one or less was five points, a case where the number is two or more and five or less was three points, and a case where the number is six or more was 0 points. These scores are shown in Table 1.

The electrolyte film-catalyst layer formation transfer film stack was rolled with a radius of 80 mm and a contact angle of 90 degrees and was delivered, and external torsional force was applied to the catalyst layer formation transfer film. The presence or absence of a flake and a breakage of 1 mm or more in the catalyst layer formation transfer film having received the external torsional force was checked. As a result, a case where the number of flakes and breakages per 100 cm² is one or less was five points, a case where the number is two or more and five or less was three points, and a case where the number is six or more was 0 points. These scores are shown in Table 1.

The total of the score of the catalyst layer formation transfer film immediately after transfer and the score of the catalyst layer formation transfer film having received the external torsional force was obtained. A case where the total score is eight points or more was A, a case where the total score is five points or more and six points or less was B, and a case where the total score is three points or less was C. Results are shown in Table 1.

TABLE 1

| | Evaluation of Catalyst Layer | | | Evaluation of Handleability Upon Transfer | | | |
|---|---|---|---|---|---|---|---|
| | Formation Transfer Film of | | | | Score of Catalyst | | |
| | Transfer Material | | | Score of Catalyst | Layer Formation | | |
| | | 90-Degree Peel Test | | Layer Formation | Transfer Film | | |
| | Maximum | Peel | | Transfer Film | Having Received | | |
| | Horizontal | Strength | | Immediately | External | Total | Overall |
| | Force (N) | (N/cm) | Peel Mode | After Transfer | Torsional Force | Score | Evaluation |
| Sample 1 | 0.41 | 0.09 | Interfacial | 5 | 5 | 10 | A |
| | | | Peeling | | | | |
| Sample 2 | 0.29 | 0.05 | Interfacial | 3 | 3 | 6 | B |
| | | | Peeling | | | | |
| Sample 3 | 0.28 | 0.04 | Interfacial | 3 | 3 | 6 | B |
| | | | Peeling | | | | |
| Sample 4 | 0.18 | 0.02 | Interfacial | 0 | 0 | 0 | C |
| | | | Peeling | | | | |

The results of Table 1 show that the maximum value of the horizontal force measured by the SAICAS method and the handleability upon transfer highly correlate with each other.

### EXPLANATION OF REFERENCE NUMERALS

10 Transfer Material
11 Catalyst Layer Formation Transfer Film
12 Base
15 Breakage
20 Cutting Blade
21 Tip End

What is claimed is:

1. A method for evaluating a catalyst layer formation transfer film stacked on a base and transferred to form a catalyst layer of a fuel cell, comprising:
measuring a horizontal force by a SAICAS method while moving a cutting blade; and
obtaining a maximum value of the horizontal force until the catalyst layer formation transfer film is broken after the cutting blade has been inserted into the catalyst layer formation transfer film and evaluating handleability of the catalyst layer formation transfer film upon transfer of the catalyst layer formation transfer film based on the maximum value of the horizontal force.

2. The method for evaluating the catalyst layer formation transfer film according to claim 1, wherein evaluating the handleability upon the transfer includes an assessment of a presence or absence of a flake and a breakage of the catalyst layer formation transfer film immediately after the catalyst layer formation transfer film has been transferred onto an electrolyte film of the fuel cell.

3. The method for evaluating the catalyst layer formation transfer film according to claim 2, wherein evaluating the handleability upon the transfer further includes an assessment of a presence or absence of a flake and a breakage of the catalyst layer formation transfer film when an external torsional force is applied to the catalyst layer formation transfer film.
